(19) United States
(12) Patent Application Publication
(10) Pub. No.: US 2023/0149579 A1
(43) Pub. Date: May 18, 2023

(54) SYSTEM AND METHOD FOR A SAFE ULTRAVIOLET DISINFECTANT DEVICE USING HUMAN PROXIMITY SENSING

(71) Applicant: Himamshu Gopalakrishna Khasnis, Bangalore (IN)

(72) Inventor: Himamshu Gopalakrishna Khasnis, Bangalore (IN)

(21) Appl. No.: 17/916,058

(22) PCT Filed: Mar. 31, 2021
(86) PCT No.: PCT/IN2021/050327

§ 371 Date: Sep. 30, 2022

(30) Foreign Application Priority Data
Mar. 31, 2020 (IN) 202041014472

(51) Int. Cl. A61L 2/24; A61L 2/10

(52) U.S. Cl. CPC A61L 2/24; A61L 2/10; A61L 2202/14; A61L 2202/11

(57) ABSTRACT

A system and method for a safe ultraviolet disinfectant device (200) using human proximity sensing is provided. The method includes capturing a proximity region of a safe UV disinfectant device (200) using a camera (302) or a human proximity sensor (202), detecting a presence of a one or more human beings from the proximity region using a human presence detection logic, controlling a power switch (508) of the safe UV disinfectant device (200) based on the presence of the one or more human beings, and causing a UV light source (204) of the safe UV disinfectant device (200) to either turn off or on to provide safe operation of the safe UV disinfectant device (200).

### CROSS-REFERENCE TO RELATED APPLICATIONS

This patent application claims priority to Indian provisional patent application no: 202041014472 filed on Mar. 31, 2021, the complete disclosures of which, in their entirety, are hereby incorporated by reference.

### BACKGROUND

### Technical Field

The embodiments herein generally relate to safe usage of an ultraviolet disinfectant, and more particularly, using human proximity sensing to provide safe usage of an ultraviolet disinfectant device.

### Description of the Related Art

Ultraviolet (UV) light in a wavelength range of 100 nano-meter (NM) to 280 NM is called a UV C light. The UV C light is known to have germicidal properties and also has an ability to kill or inactivate micro-organisms by destroying nucleic acids and disrupting their DNA. This leaves the micro-organisms unable to perform vital cellular functions and a surface to which is exposed to the UV C light is disinfected. In the event of pandemics like 2019-20 COVID-19, the properties of the UV C light are utilized to disinfect surfaces and materials thus helping reduction of virus spread.

Traditionally, there have been UV light emitting handheld wands proposed in the past for this purpose. UV wand built can be used to sanitize any surface as it kills epidemic micro-organisms such as Escherichia coli (E-coli), Salmonella, typhii, Staphylococcus aureus, avian bird flu and SARS virus with a high killing percentage of 99.99% in 10 seconds. However, since human beings are vulnerable to UV light exposure, there is a ubiquitous worry about accidental UV light exposure to humans, which causes a depreciation in the widespread usage of UV light based disinfectant devices. An exposure of UV light to a human being is harmful and may cause damage to eye-sight, skin burns. A long term exposure may even lead to cancer development in a human-being.

FIG. 1 illustrates a traditional (Prior art) ultraviolet (UV) disinfectant device 100 according to some embodiments herein. The traditional the UV disinfectant device 100 includes a UV light source 102, a transparent window 104 and an opaque UV resistant enclosure 106. In some embodiments, the traditional the UV disinfectant device 100 may be a hand-held device. In some embodiments, the safe UV disinfectant device 200 is held using the opaque UV resistant enclosure 108. In some embodiments, a switch may be operated by a user that causes the UV light source 102 to turn on. The UV light source 102 emits a UV light that passes through the transparent window 104. In some embodiments, the UV light strikes on a desired surface and causes disinfection of the desired surface by killing micro-organisms on the desired surface.

Accordingly, there remains a need of safe to use UV disinfectant device that automatically detects proximity of a human being and provides safe operation of the UV disinfectant device.

### SUMMARY

In view of the foregoing, embodiments herein provide a system of a safe ultraviolet (UV) disinfectant device using human proximity sensing. The system includes (a) a human proximity sensor that detects a presence of nearby objects, (b) an ultraviolet light source that emits a UV light on a desired surface, and (c) a control unit that switches the ultraviolet light source on or off by (i) capturing a proximity region of the safe UV disinfectant device using the human proximity sensor, (ii) detecting the presence of at least one human being from the proximity region using image recognition, and (iii) disabling a power switch based on the presence of at least one human being to provide safe operation of the safe UV disinfectant device (200).

In some embodiments, the human proximity sensor is an image capturing device that continuously captures images of a long range proximity region of the safe UV disinfectant device.

In some embodiments, the human proximity sensor continuously captures images of a short range proximity region of the safe UV disinfectant device.

In some embodiments, the safe UV disinfectant device includes a thermal or capacitive sensor that detects a part of the at least one human being.

In some embodiments, the safe UV disinfectant device includes an opaque UV resistant enclosure below a transparent window that encloses the ultraviolet light source for enabling a hand-held wand operation of the safe UV disinfectant device.

In another aspect, there is provided a method of safe operation of a safe ultraviolet (UV) disinfectant device using human proximity sensing. The method includes (i) capturing a proximity region of a safe UV disinfectant device using a human proximity sensor, (ii) emitting UV light on a desired surface from a UV light source, (iii) detecting a presence of at least one human being from the proximity region using a human presence detection logic using image recognition, and (iv) disabling a power switch of the safe UV disinfectant device based on the presence of at least one human being to provide safe operation of the safe UV disinfectant device.

In some embodiments, the human proximity sensor is an image capturing device that continuously captures images of a long range proximity region of the safe UV disinfectant device (200).

In some embodiments, the human proximity sensor continuously captures images of a short range proximity region of the safe UV disinfectant device.

In some embodiments, the safe UV disinfectant device includes a thermal or capacitive sensor that detects a part of the at least one human being.

In some embodiments, the safe UV disinfectant device includes an opaque UV resistant enclosure below a transparent window that encloses the ultraviolet light source for enabling a hand-held wand operation of the safe UV disinfectant device.

### BRIEF DESCRIPTION OF THE DRAWINGS

The embodiments herein will be better understood from the following detailed description with reference to the drawings, in which:

FIG. 1 illustrates a traditional (Prior art) ultraviolet (UV) disinfectant device according to some embodiments herein;

FIG. 2 illustrates a safe UV disinfectant device with a human proximity sensor according to some embodiments herein;

FIG. 3 illustrates the safe UV disinfectant device with a camera according to some embodiments herein;

FIG. 4 illustrates the safe UV disinfectant device with the human proximity sensor and the camera according to some embodiments herein;

FIG. 5 is block diagram of a control unit of the safe UV disinfectant device according to some embodiments herein;

FIG. 6 is a flow chart that illustrates a method of performing safe ultraviolet disinfection using human proximity sensing and the camera; and

FIG. 7 is a representative hardware environment for practicing the embodiments herein with respect to FIG. 1 through 6.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

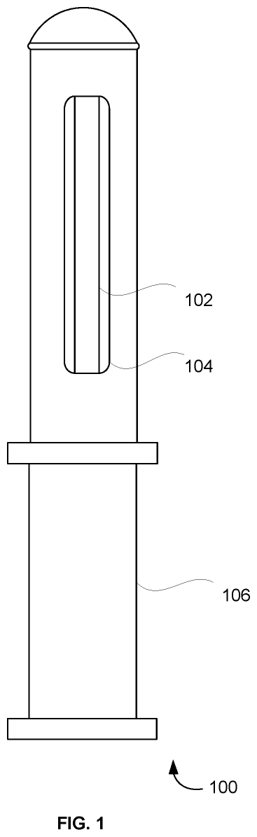

In the view of the foregoing, the need of a UV disinfectant device that is safe to use that automatically detects proximity of a human being and provides safe operation of the UV disinfectant device is fulfilled in the ongoing description by: (a) capturing a proximity region of a safe UV disinfectant device using a camera or a human proximity sensor, (b) detecting a presence of a one or more human beings from the proximity region using a human presence detection logic, (c) controlling a power switch of the safe UV disinfectant device based on the presence of the one or more human beings, and (d) causing a UV light source of the safe UV disinfectant device to either turn off or on to provide safe operation of the safe UV disinfectant device. In some embodiments, the safe UV disinfectant device is implemented in a wand like object. Referring now to the drawings, and more particularly to FIGS. 1 through 6, where similar reference characters denote corresponding features in a consistent manner throughout the figures, there are shown preferred embodiments.

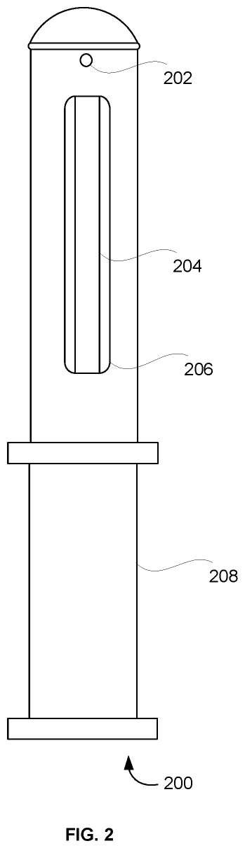

FIG. 2 illustrates a safe UV disinfectant device 200 with a human proximity sensor according to some embodiments herein. The safe UV disinfectant device 200 includes a human proximity sensor 202, a UV light source 204, a transparent window 206 and an opaque UV resistant enclosure 208. In some embodiments, the safe UV disinfectant device 200 may be a hand-held device or a wand. In some embodiments, the safe UV disinfectant device 200 may be held using the opaque UV resistant enclosure 208. The UV light source 204 emits a UV light that passes through the transparent window 206. In some embodiments, the UV light strikes on the desired surface and causes disinfection of the desired surface by killing micro-organisms on the desired surface. In some embodiments, the human proximity sensor 202 capturing a proximity region of a safe UV disinfectant device. The proximity region captured from the human proximity sensor 202 may be utilized to detect a presence of a one or more human beings in a proximity of the safe UV disinfectant device 200. In some embodiments, when a one or more human beings is detected by the safe UV disinfectant device 200, the UV light source 204 of the safe UV disinfectant device 200 may be switched on or off. In some embodiments, the human proximity sensor 202 may actively sense for the presence of the one or more human beings throughout the operation of UV lights. In some embodiments, the safe UV disinfectant device 200 includes a control unit that is configured to (i) capture a proximity region of the safe UV disinfectant device (200) using the human proximity 202, (ii) detect a presence of at least one human being from the proximity region using image recognition and (iii) disable a power switch based on the presence of at least one human being to provide safe operation of the safe UV disinfectant device (200).

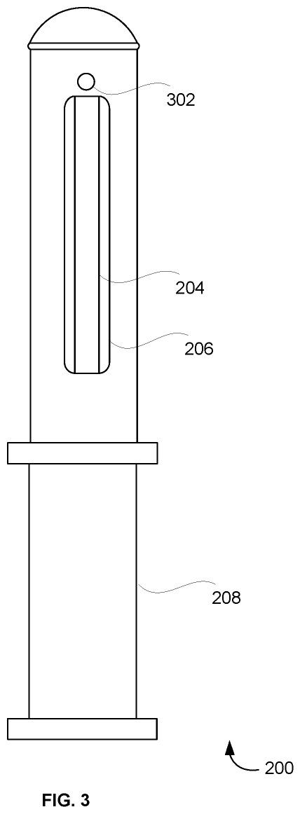

With reference to FIG. 2, FIG. 3 illustrates the safe UV disinfectant device with a camera according to some embodiments herein. The safe UV disinfectant device 200 includes a camera 302, the UV light source 204, the transparent window 206 and the opaque UV resistant enclosure 208. In some embodiments, the safe UV disinfectant device 200 may be a hand-held device or a wand. In some embodiments, the safe UV disinfectant device 200 may be held using the opaque UV resistant enclosure 208. The UV light source 204 emits a UV light that passes through the transparent window 206. In some embodiments, the UV light strikes on the desired surface and causes disinfection of the desired surface by killing micro-organisms on the desired surface. In some embodiments, the camera 302 capturing an image of the proximity region of the safe UV disinfectant device 200. The proximity region captured from the human proximity sensor 202 may be utilized to detect a presence of a one or more human beings in a proximity of the safe UV disinfectant device 200. In some embodiments, when a one or more human beings is detected by the safe UV disinfectant device 200, the UV light source 204 of the safe UV disinfectant device 200 may be switched on or off. In some embodiments, the camera 302 utilizes an image recognition algorithm to detect the presence of the one or more human beings in a field of UV light.

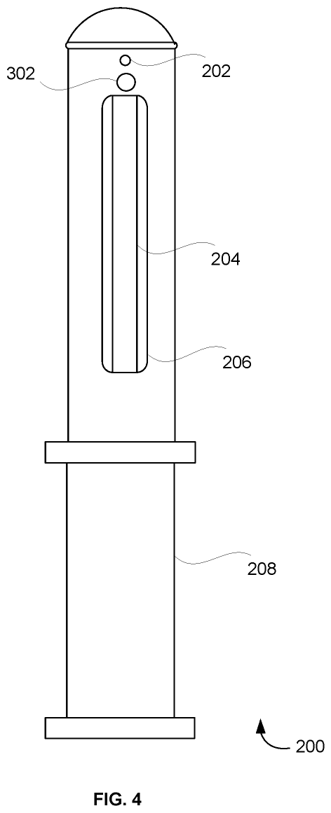

With reference to FIG. 2, FIG. 4 illustrates the safe UV disinfectant device with the human proximity sensor 202 and the camera 302 according to some embodiments herein. The safe UV disinfectant device 200 includes the human proximity sensor 202, the camera 302, the UV light source 204, the transparent window 206 and the opaque UV resistant enclosure 208. In some embodiments, the safe UV disinfectant device 200 may be a hand-held device or a wand. In some embodiments, the safe UV disinfectant device 200 may be held using the opaque UV resistant enclosure 208. The UV light source 204 emits a UV light that passes through the transparent window 206. In some embodiments, the UV light strikes on the desired surface and causes disinfection of the desired surface by killing micro-organisms on the desired surface. In some embodiments, the camera 302 capturing an image of the proximity region of the safe UV disinfectant device 200. The proximity region captured from the human proximity sensor 202 may be utilized to detect a presence of a one or more human beings in a proximity of the safe UV disinfectant device 200. In some embodiments, when a one or more human beings is detected by the safe UV disinfectant device 200, the UV light source 204 of the safe UV disinfectant device 200 may be switched on or off. In some embodiments, when the wand is too close to a human body part, visual features of the one or more human beings near the wand may be obscure. The embodiment of FIG. 4 provides safest methods of sensing human presence as utilizing both the human proximity sensor 202 and the camera 302 covers failure-cases of each other. In an exemplary embodiment, when the human proximity sensor 202 fails to detect the presence of the one or more human beings, the camera 302 is able to detect the presence of the one or more human beings and vice-versa.

In some embodiments, the safe UV disinfectant device 200 may include sensors like thermal sensors, capacitive sensors that provide better detection of the presence of the one or more human beings when the distance between the one or more human beings and the safe UV disinfectant device 200 is less. In some embodiments, when the distance between the one or more human beings and the safe UV disinfectant device 200 is more, the human presence detection module 506 utilizes the camera 302 to detect the presence of the one or more human beings.

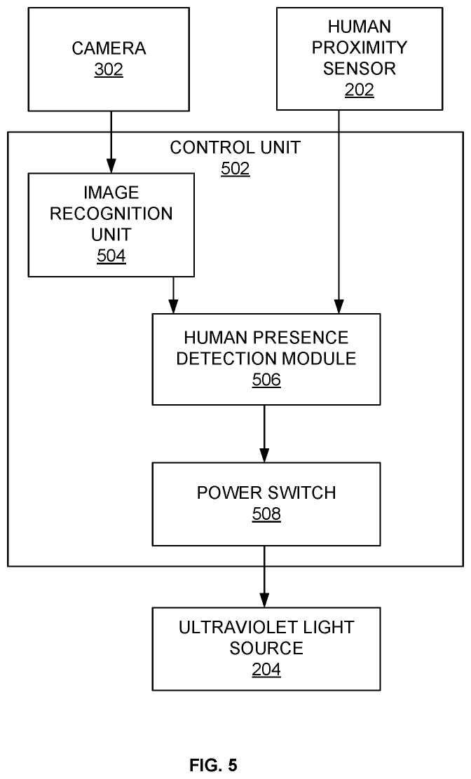

With reference to FIG. 2, FIG. 5 is block diagram of a control unit of the safe UV disinfectant device (200) according to some embodiments herein. The control unit 502 includes an image recognition unit 504, a human presence detection module 506 and a power switch 508. In some embodiments, the image recognition unit 504 is connected to the camera 302 of the safe UV disinfectant device 200. In some embodiments, the human presence detection module 506 is connected to the human proximity sensor 202 of the safe UV disinfectant device 200. In some embodiments, the human presence detection module 506 is connected to both the image recognition unit 504 and the human presence detection module 506. The human presence detection module 506 detects the presence of a one or more human beings in the proximity of the safe UV disinfectant device 200 and controls the power switch 508 of the UV light source 204. The power switch 508 is connected to the UV light source 204. In some embodiments, the human presence detection module 506 includes image recognition algorithms.

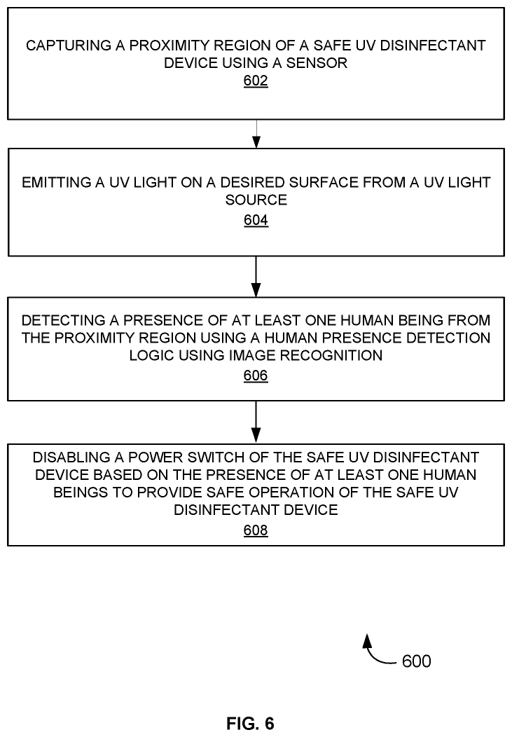

FIG. 6 is a flow chart that illustrates a method of performing safe ultraviolet disinfection using human proximity sensing. At step 602, the method includes capturing a proximity region of a safe UV disinfectant device using a sensor. At step 604, the method includes emitting a UV light on a desired surface from a UV light source. At step 606, the method includes detecting a presence of at least one human being from the proximity region using a human presence detection logic using image recognition. At step 608, the method includes disabling a power switch of the safe UV disinfectant device based on the presence of at least one human being to provide safe operation of the safe UV disinfectant device.

The embodiments herein may include a computer program product configured to include a pre-configured set of instructions, which when performed, can result in actions as stated in conjunction with the methods described above. In an example, the pre-configured set of instructions can be stored on a tangible non-transitory computer readable medium or a program storage device. In an example, the tangible non-transitory computer readable medium can be configured to include the set of instructions, which when performed by a device, can cause the device to perform acts similar to the ones described here. Embodiments herein may also include tangible and/or non-transitory computer-readable storage media for carrying or having computer executable instructions or data structures stored thereon.

Generally, program modules utilized herein include routines, programs, components, data structures, objects, and the functions inherent in the design of special-purpose processors, etc. that perform particular tasks or implement particular abstract data types. Computer executable instructions, associated data structures, and program modules represent examples of the program code means for executing steps of the methods disclosed herein. The particular sequence of such executable instructions or associated data structures represents examples of corresponding acts for implementing the functions described in such steps.

The embodiments herein can include both hardware and software elements. The embodiments that are implemented in software include but are not limited to, firmware, resident software, microcode, etc.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

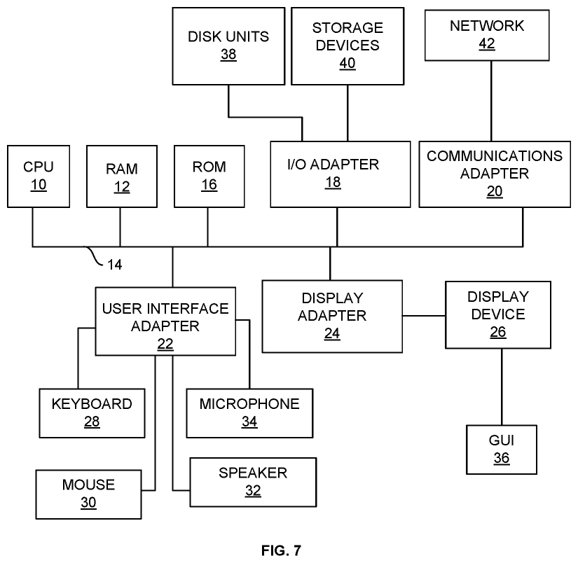

A representative hardware environment for practicing the embodiments herein is depicted in FIG. 7, with reference to FIGS. 1 through 6. This schematic drawing illustrates a hardware configuration of a server/computer system/ computing device in accordance with the embodiments herein. The system includes at least one processing device CPU 10 that may be interconnected via system bus 14 to various devices such as a random access memory (RAM) 12, read-only memory (ROM) 16, and an input/output (I/O) adapter 18. The I/O adapter 18 can connect to peripheral devices, such as disk units 38 and program storage devices 40 that are readable by the system. The system can read the inventive instructions on the program storage devices 40 and follow these instructions to execute the methodology of the embodiments herein. The system further includes a user interface adapter 22 that connects a keyboard 28, mouse 30, speaker 32, microphone 34, and/or other user interface devices such as a touch screen device (not shown) to the bus 14 to gather user input. Additionally, a communication adapter 20 connects the bus 14 to a data processing network 42, and a display adapter 24 connects the bus 14 to a display device 26, which provides a graphical user interface (GUI) 36 of the output data in accordance with the embodiments herein, or which may be embodied as an output device such as a monitor, printer, or transmitter, for example.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation.

What is claimed is:

1. A system of a safe ultraviolet (UV) disinfectant device using human proximity sensing, the system comprising:
a human proximity sensor that detects a presence of nearby objects;
an ultraviolet light source that emits a UV light on a desired surface; and
a control unit that switches the ultraviolet light source on or off by:
capturing a proximity region of the safe UV disinfectant device using the human proximity sensor;
detecting a presence of at least one human being from the proximity region using image recognition; and
disabling a power switch based on the presence of at least one human being to provide safe operation of the safe UV disinfectant device.

2. The system of claim 1, wherein the human proximity sensor is an image capturing device that continuously captures images of a long range proximity region of the safe UV disinfectant device.

3. The system of claim 2, wherein the human proximity sensor continuously captures images of a short range proximity region of the safe UV disinfectant device.

4. The system of claim 3, wherein the safe UV disinfectant device includes a thermal or capacitive sensor that detects a part of the at least one human being.

5. The system of claim 4, wherein the safe UV disinfectant device includes an opaque UV resistant enclosure below a transparent window that encloses the ultraviolet light source for enabling a hand-held wand operation of the safe UV disinfectant device.

6. A method of safe operation of a safe ultraviolet (UV) disinfectant device using human proximity sensing, the method comprising:
capturing a proximity region of a safe UV disinfectant device using a human proximity sensor;
emitting UV light on a desired surface from a UV light source;
detecting a presence of at least one human being from the proximity region using a human presence detection logic using image recognition; and
disabling a power switch of the safe UV disinfectant device based on the presence of at least one human being to provide safe operation of the safe UV disinfectant device.

7. The method of claim 6, wherein the human proximity sensor is an image capturing device that continuously captures images of a long range proximity region of the safe UV disinfectant device.

8. The method of claim 7, wherein the human proximity sensor continuously captures images of a short range proximity region of the safe UV disinfectant device.

9. The method of claim 8, wherein the safe UV disinfectant device includes a thermal or capacitive sensor that detects a part of the at least one human being.

10. The method of clim 9, wherein the safe UV disinfectant device includes an opaque UV resistant enclosure below a transparent window that encloses the ultraviolet light source for enabling a hand-held wand operation of the safe UV disinfectant device.
